(19) United States
(12) Patent Application Publication
(10) Pub. No.: US 2014/0288441 A1
(43) Pub. Date: Sep. 25, 2014

(54) SENSING PHYSIOLOGICAL CHARACTERISTICS IN ASSOCIATION WITH EAR-RELATED DEVICES OR IMPLEMENTS

(71) Applicants: Michael Edward Smith Luna, San Jose, CA (US); Thomas Alan Donaldson, Nailsworth (GB); Scott Fullam, Palo Alto, CA (US)

(72) Inventors: Michael Edward Smith Luna, San Jose, CA (US); Thomas Alan Donaldson, Nailsworth (GB); Scott Fullam, Palo Alto, CA (US)

(73) Assignee: AliphCom, San Francisco, CA (US)

(21) Appl. No.: 14/207,183

(22) Filed: Mar. 12, 2014

Related U.S. Application Data
(60) Provisional application No. 61/785,743, filed on Mar. 14, 2013.

(51) Int. Cl. A61B 5/00; A61B 7/04; A61B 5/0205

(52) U.S. Cl. CPC A61B 5/6815; A61B 5/6838; A61B 5/0004; A61B 5/0205; A61B 7/04; A61B 5/053
USPC 600/484; 600/300; 600/547; 600/509; 600/529; 600/485; 600/586

(57) ABSTRACT

Various embodiments relate generally to electrical and electronic hardware, computer software, wired and wireless network communications, and wearable computing and audio devices for monitoring health and wellness. More specifically, disclosed are an apparatus and a method for processing signals representing physiological characteristics sensed from tissue at or adjacent an ear of an organism. In one or more embodiments, a wearable device includes a sensor terminal and a physiological sensor coupled to the sensor terminal to sense one or more signals originating at the sensor terminal. The wearable device may also include a radio frequency (“RF”) communications interface. Also, the wearable device can include a processor configured to cause generation of data representing a physiological characteristic of the organism.

### CROSS-REFERENCE TO RELATED APPLICATIONS

This application is a U.S. non-provisional patent application that claims the benefit of U.S. Provisional Patent Application No. 61/785,743, filed Mar. 14, 2013, and entitled “SENSING PHYSIOLOGICAL CHARACTERISTICS IN ASSOCIATION WITH EAR-RELATED DEVICES OR IMPLEMENTS,” which is herein incorporated by reference for all purposes.

### FIELD

Various embodiments relate generally to electrical and electronic hardware, computer software, wired and wireless network communications, and wearable computing and audio devices for monitoring health and wellness. More specifically, disclosed are an apparatus and a method for processing signals representing physiological characteristics sensed from tissue at or adjacent an ear of an organism.

### BACKGROUND

Conventional techniques for acquiring physiological information from an organism, such as a human, typically required the assistance of trained medical personnel. While there exists some devices and techniques for a layperson to determine physiological characteristics, such as heart rate, such devices are not well-suited for everyday activities of active people. Typical devices for determining physiological characteristics are typically designed to attach to a proximal portion of a limb, such as an upper arm, or about or on the chest of the user.

Thus, what is needed is a solution for data capture devices, such as for wearable devices, without the limitations of conventional techniques.

### BRIEF DESCRIPTION OF THE DRAWINGS

Various embodiments or examples (“examples”) of the invention are disclosed in the following detailed description and the accompanying drawings:

FIG. 1 illustrates an example of an implementation of the ear-related device/implement configured to facilitate sensing of physiological signals, according to some embodiments;

FIG. 2 depicts an ear-related device/implement configured to receive signals describing physiological characteristics, according to some embodiments;

FIG. 3 depicts another example of an ear-related device/implement configured to provide for sensor terminals to sense physiological characteristics, according to some embodiments;

FIG. 4 depicts perspective and top views of the ear-related device/implement shown in FIG. 3, according to some examples;

FIG. 5 depicts another example of an ear-related device/implement configured to provide for sensor terminals to sense physiological characteristics, according to some embodiments;

FIG. 6 depicts another example of an ear-related device/implement configured to provide for sensor terminals to sense physiological characteristics, according to some embodiments;

FIG. 7 depicts another example of an ear-related device/implement configured to provide sensor terminals to sense physiological characteristics, according to some embodiments;

FIG. 8 depicts yet another example of an ear-related device/implement configured to provide sensor terminals to sense physiological characteristics, according some embodiments; and

FIG. 9 illustrates an exemplary computing platform disposed in or otherwise associated with an ear-related device/implement in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

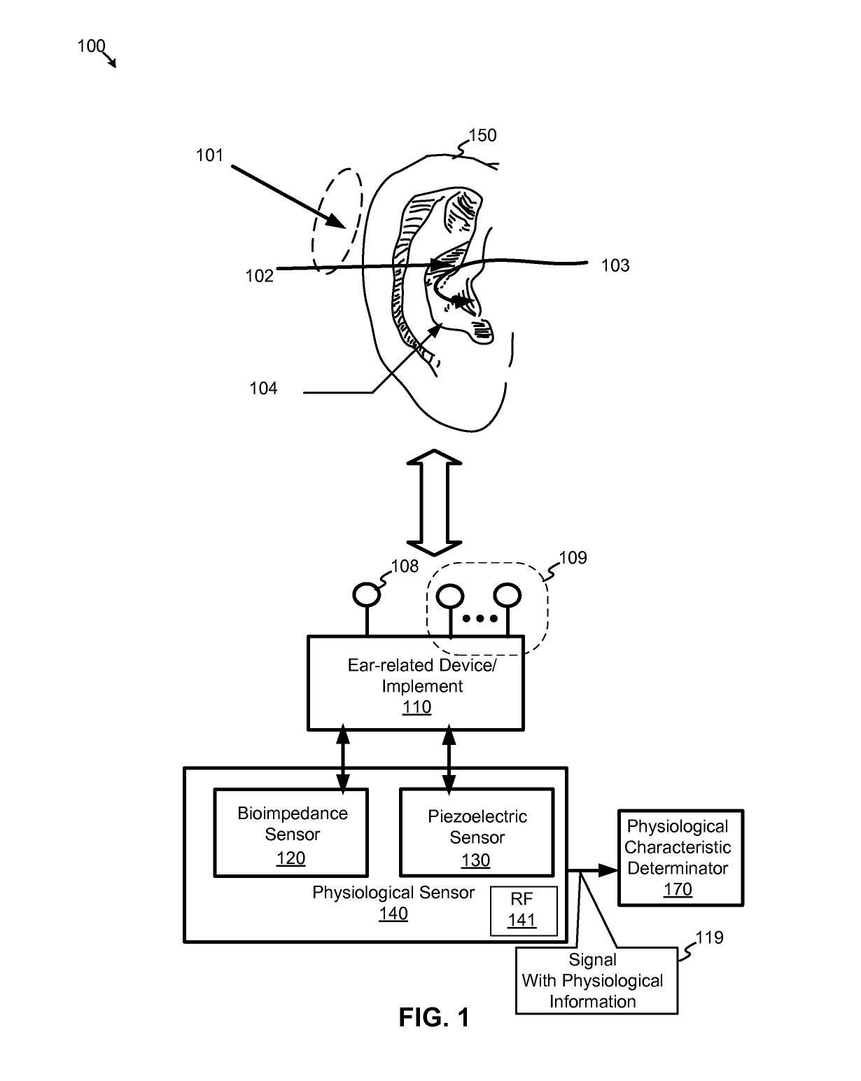

FIG. 1 illustrates an example of an implementation of the ear related device/implement configured to facilitate sensing of physiological signals, according to some embodiments. Diagram 100 depicts an ear-related device/implement 110 coupled to at least one sensor terminal 108. In some examples, ear-related device/implement 110 can be coupled to multiple sensor terminals including sensor terminal 108 and any number of sensor terminals 109. As shown, any of the sensor terminals 108 and 109 can be positioned to sense physiological signals from or at various portions of tissue at or near ear 150 or regions thereabout, such as a region of skin 101, which is behind the ear 150. In one example, a sensor terminal can be positioned adjacent a cymba concha 102 portion of ear 150. In another example, a sensor terminal can be positioned adjacent a portion of a targus region 103 of ear 150. In still another example, a sensor terminal can be position adjacent to a portion of a cymba cavum region 104 of ear 150. Also, the sensor terminal can be disposed adjacent a region of tissue 101. Sensor terminals 108 and are not limited to sensing physiological signals from the above-identified regions, but rather can sense physiological signals from any part of ear 150. Examples of ear-related device/implement 110 include headsets (e.g., Bluetooth® headsets), headphones (e.g., wireless headphones), and any other device. For example, ear-related device/implement 110 can include or be disposed in the speaker portion of a mobile computing device or mobile phone, or any other device configured to, for instance, provide audio or facilitate in uni- or bi-directional communications. In some cases, ear-related device/implement 110 can include implements such as eyewear (e.g., including the portions that extend behind an ear), hats (e.g., including those portions that extend behind or over an ear), earbuds, or any other instrument or implement upon which at least sensor terminals can be disposed.

Diagram 100 depicts a physiological sensor 140 configured to generate one or more physiological signals that can be used to derive physiological signals, such as heart rate, respiration, and other detectable physiological characteristics, for example, from the sensor terminals. Any ear-related device can include a physiological sensor 140 and a physiological characteristic determinator 170, which can be implemented as a physiological signal generator in some embodiments. Physiological sensor 140 can be configured to sense signals, such as physiological signals, associated with a physiological characteristic.

Ear-related device/implement 110 can coupled to or can include a physiological sensor 140 and/or a physiological characteristic determinator 170. Physiological sensor 140 is configured to receive the sensed signals from one or more of the sensor terminal 108 and/or any of sensor terminals 109. In one embodiment, physiological sensor 140 includes a bioimpedance sensor 120. In some embodiments, sensor terminal 108 and/or any of sensor terminals 109 are electrodes coupled to bioimpedance sensor 120, which is configured to determine the bioelectric impedance (“bioimpedance”) of one or more types of tissues of a wearer to identify, measure, and monitor physiological characteristics. For example, a drive signal having a known amplitude and frequency can be applied to a user, from which a sink signal is received as bioimpedance signal. The bioimpedance signal is a measured signal that includes real and complex components. Examples of real components include extra-cellular and intra-cellular spaces of tissue, among other things, and examples of complex components include cellular membrane capacitance, among other things. Further, the measured bioimpedance signal can include real and/or complex components associated with arterial structures (e.g., arterial cells, etc.) and the presence (or absence) of blood pulsing through an arterial structure. In some examples, a heart rate signal, or other physiological signals, can be determined (i.e., recovered) from the measured bioimpedance signal by, for example, comparing the measured bioimpedance signal against the waveform of the drive signal to determine a phase delay (or shift) of the measured complex components. The bioimpedance sensor signals can provide a heart rate, a respiration rate, and a Mayer wave rate. Non-limiting examples of a bioimpedance sensor and a physiological characteristic determinator are described in U.S. patent application Ser. No. 13/802,319, filed on Mar. 13, 2013, which is herein incorporated herein by reference. Further, multiple sensor terminals 108 and 109 can contact a common portion of ear 150 (e.g., two sensor terminals can extract a bioimpedance signal from cymba concha 102 portion of ear 150). In other instances, one or more sensor terminals can extract a bioimpedance signal from two or more regions (e.g., an AC signal can be injected into cymba concha 102 portion of ear 150 and extracted from cymba cavum region 104 of ear 150).

In some embodiments, physiological sensor 140 includes a piezoelectric sensing element as a sensor terminal 108. In this case, sensor terminal 108 can be configured to sense, for example, acoustic energy and to generate an electric signal indicative to the characteristics of the acoustic energy. Sensor terminal 108 (as well as other sensor terminals 109) can be positioned adjacent to a source of physiological signals, such as adjacent to a blood vessel. According to some embodiments, physiological sensor 140 is a piezoelectric sensor 170 (e.g., a portion of which is a piezoelectric transducer) configured to receive, for example, acoustic energy, and further configured to generate piezoelectric signals (e.g., electrical signals). In the example shown, piezoelectric sensor 130 is configured to receive an acoustic signal that includes, for example, heart-related signals. For example, an acoustic signal can propagate through at least human tissue as sound energy waveforms. Such sound energy signals can originate from either a heart beating (e.g., via a blood vessel) or blood pulsing through a blood vessel, or both. The energy propagating as an acoustic signal into a sensor terminal of piezoelectric sensor 140, which is converts the acoustic energy into piezoelectric signals transmitted to physiological characteristic determinator 170. Physiological characteristic determinator 170, which, in some examples, can be described as a physiological signal generator, is configured to detect and identify, for example, heartbeats. An example of a piezoelectric sensor that can be implemented is described in U.S. patent application Ser. No. 13/672,398, filed on Nov. 8, 2012, both of which are incorporated by reference. As used herein, the term tissue can refer to, at least in some examples, as skin, muscle, blood, or other tissue.

In some embodiments, physiological sensor 130 can implement a microphone to detect acoustic energy and sound waves. A microphone (not shown) configured to contact (or to be positioned adjacent to) the skin of the wearer, whereby the microphone is adapted to receive sound and acoustic energy generated by the wearer (e.g., the source of sounds associated with physiological information). The microphone can also be disposed at the ear as a sensor terminal 108 and/or any of sensor terminal 109 (e.g., when differentially sensing acoustic signals). According to some embodiments, the microphone can be implemented as a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005, and U.S. patent application Ser. No. 13/672,398, filed on Nov. 8, 2012, both of which are incorporated by reference. As used herein, the term human tissue can refer to, at least in some examples, as skin, muscle, blood, or other tissue. Note that signal 119 can represent a raw bioimpedance signal (e.g., an electrical signal) or a piezoelectric signal (e.g., an electrical signal) that embodies data describing the physiological characteristics (i.e., some processing may be performed to extract physiological signals at physiological characteristic determinator 170). Or, signal 119 can represent the physiological signals. Note that in some embodiments, physiological signals can be related to any physiological signals (e.g., need not be limited to heart-related signals). Further, physiological sensor 140 can include a wireless transceiver (“RF”) 141 configured to transmit and receive radio frequency signals for communication physiological information, among other things.

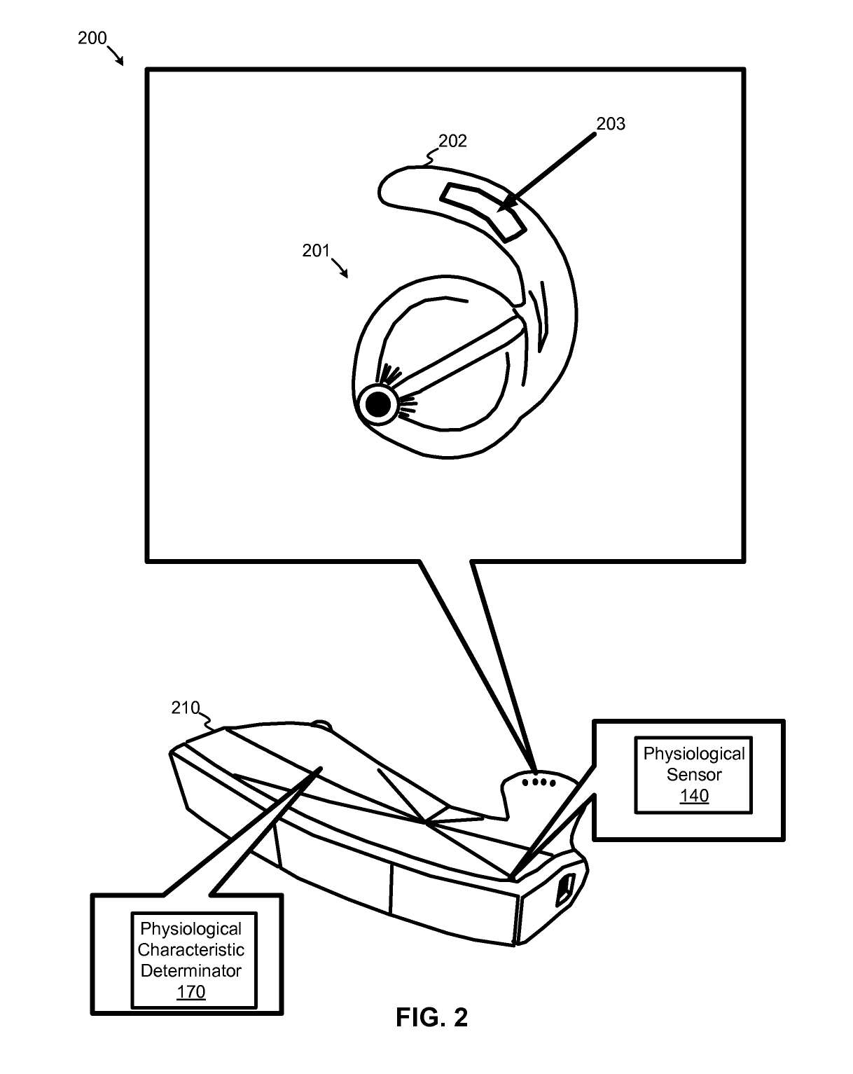

FIGS. 2 to 8 depict several examples and are not intend to be limiting. Various embodiments are broader than as described therein.

FIG. 2 depicts an ear-related device/implement configured to receive signals describing physiological characteristics, according to some embodiments. Diagram 200 depicts ear-related device/implement 210 including an earbud 201 having an extension structure 202 (e.g., a portion of a C-type earbud, such as those manufactured by Jawbone®) that includes one or more sensor terminals 203. In some examples, sensor terminals 203 are conductive and can be configured to apply and/or receive a bioimpedance signal. Such a signal can be received by ear-related device/implement 210 which includes at least physiological sensor 140. In some examples, sensor terminal 203 can be a piezoelectric transducer or related structures. Thus, physiological sensor 140 can be a bioimpedance sensor or an acoustic sensor, such as a piezoelectric sensor. In some examples of physiological characteristic determinator 170 can be disposed in ear-related device/implement 210, but can also be disposed in any other device, in communication with ear-related device/implement 210, such as a mobile device or phone. In some examples, extension structure 202 is configured to apply a spring-like force to a cymba concha so that sensor terminal 203 is in contact with tissue. In some cases extension structure 202 is configured to minimize vibrations (and noise associated therewith). Therefore, extension structure 202 can enhance signal quality and integrity of a sensed signal (e.g., improving a signal-to-noise ratio).

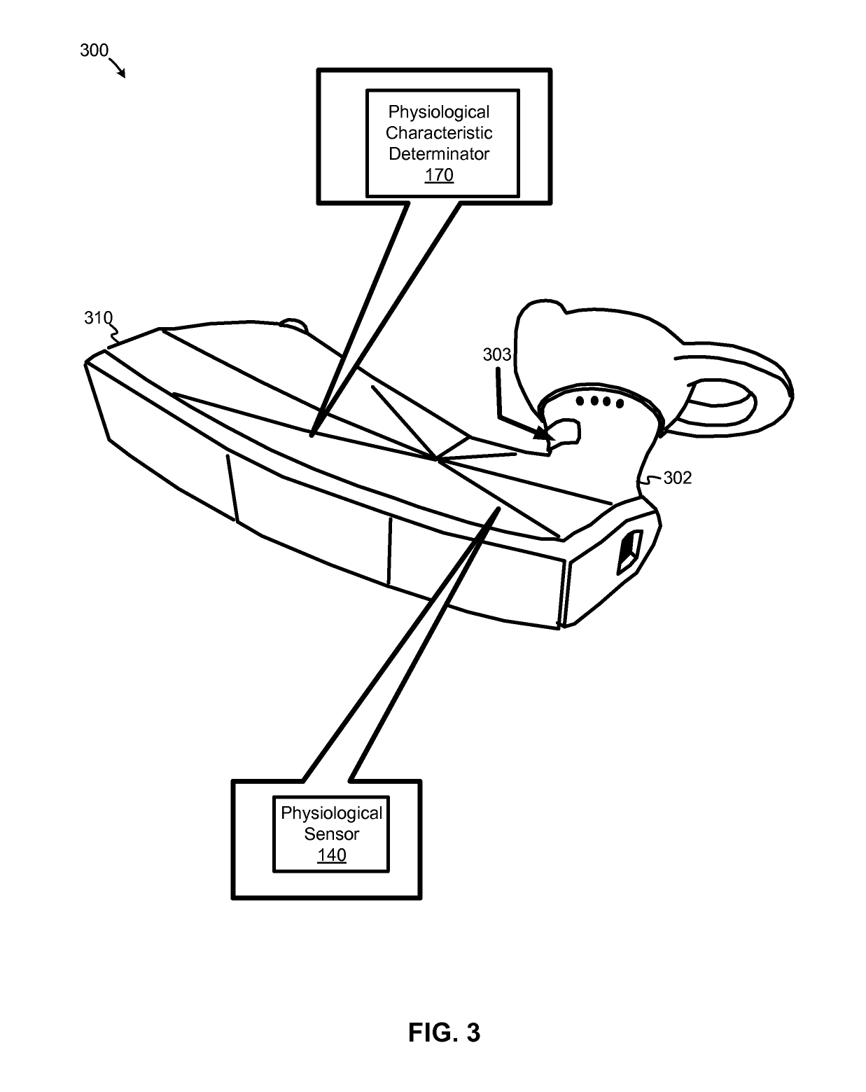

FIG. 3 depicts another example of an ear-related device/implement configured to provide for sensor terminals to sense physiological characteristics, according to some embodiments. Diagram 300 depicts ear-related device/implement 310 including a neck portion 302 that can include one or more sensor terminals 303. In some examples, sensor terminals 303 are conductive and are configured to apply and/or receive a bioimpedance signal. Such a signal can be received by ear-related device/implement 210 which includes at least physiological sensor 140. In some examples, sensor terminal 303 can be a piezoelectric transducer or related structures. Thus, physiological sensor 140 can be a bioimpedance sensor or an acoustic sensor, such as a piezoelectric sensor. In some examples of physiological characteristic determinator 170 can be disposed in ear-related device/implement 310, but can also be disposed in any other device, in communication with ear-related device/implement 310, such as a mobile device or phone (not shown). In some examples, neck portion 302 can be configured to apply a force to a portion of a targus portion (e.g., adjacent to the ear canal) inside of an ear so that sensor terminal 303 is in contact with tissue.

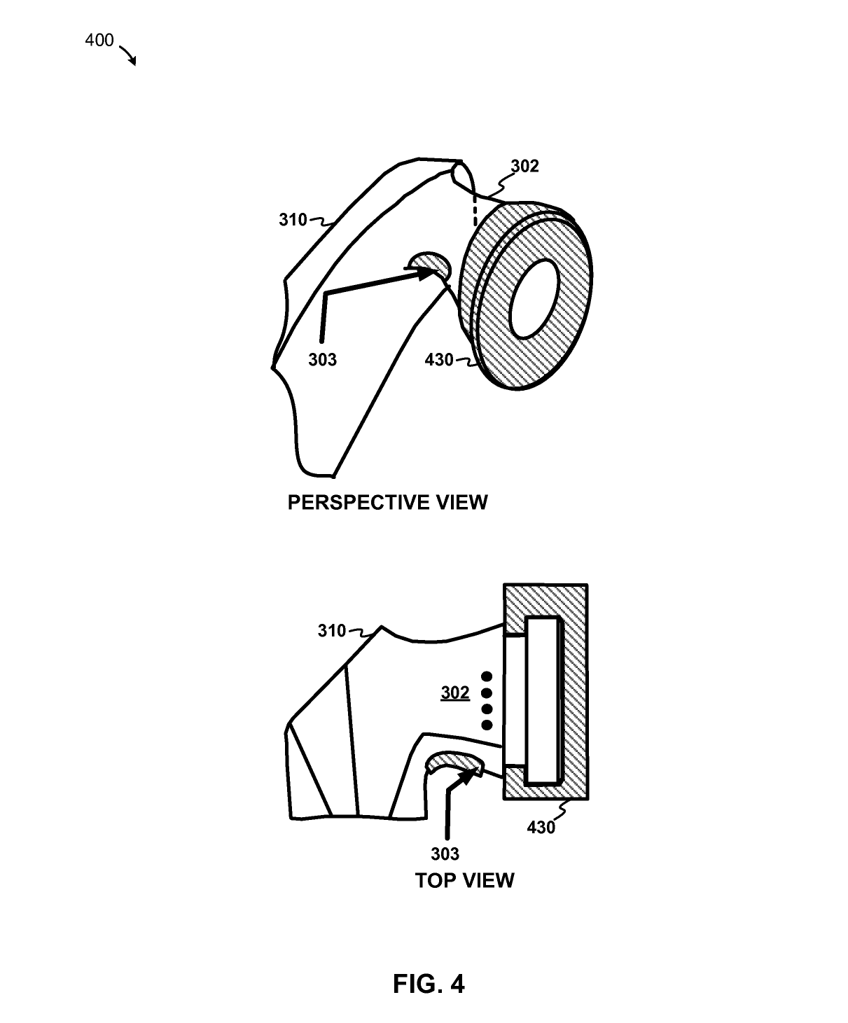

FIG. 4 depicts perspective and top views of the ear-related device/implement shown in FIG. 3, according to some examples. Diagram 400 includes a perspective view and a top view. The perspective view depicts a sensor terminal 303 co-located on neck 302, whereby an earbud 430 is configured to contact portions of an ear canal to establish relatively firm contact between source terminal 303 and the tissue of the targus. The top view depicts the positioning of source terminal 303 on neck 302, along with earbud 430. Note that multiple source terminals 303 can be implemented at different portions of 302 to contact the targus or any other ear portion at multiple points.

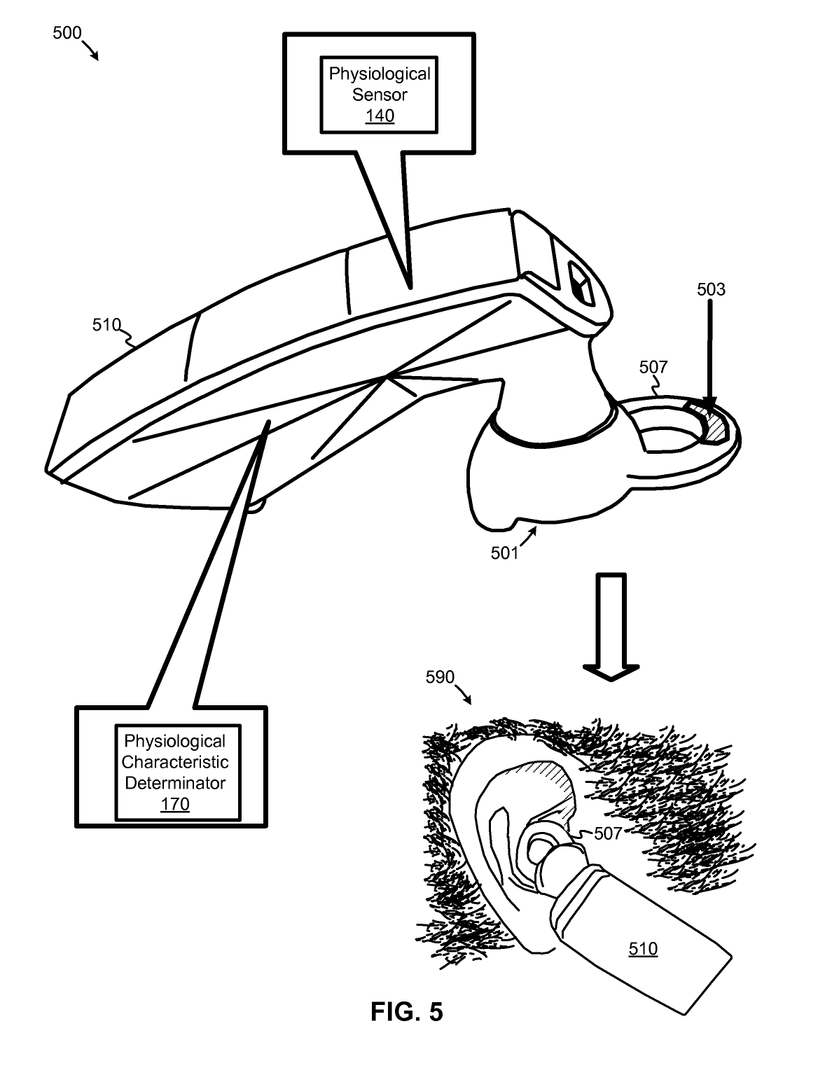

FIG. 5 depicts another example of an ear-related device/implement configured to provide for sensor terminals to sense physiological characteristics, according to some embodiments. Diagram 500 depicts ear-related device/implement 510 including an earbud 501 (e.g., a loop-spout bud) that can include one or more sensor terminals 503 disposed on or at loop portion 507. In some examples, sensor terminals 503 can be conductive and can be configured to apply and/or receive a bioimpedance signal. Such a signal can be received by ear-related device/implement 510 which includes at least physiological sensor 140. In some examples, sensor terminal 503 can be a piezoelectric transducer or related structures. Thus, physiological sensor 140 can be a bioimpedance sensor or an acoustic sensor, such as a piezoelectric sensor. In some examples of physiological characteristic determinator 170 can be disposed in ear-related device/implement 510, but can also be disposed in any other device, in communication with ear-related device/implement 510, such as a mobile device or phone (not shown). In some examples, loop portion 507 is inserted within an ear, as shown in diagram 590, whereby sensor terminal 503 can be positioned adjacent to or in contact with the concha cavum or the back of the concha. The loop portion 507 provides, at least in one example, a horizontal reaction force via the back of the concha, which can bend loop portion 507.

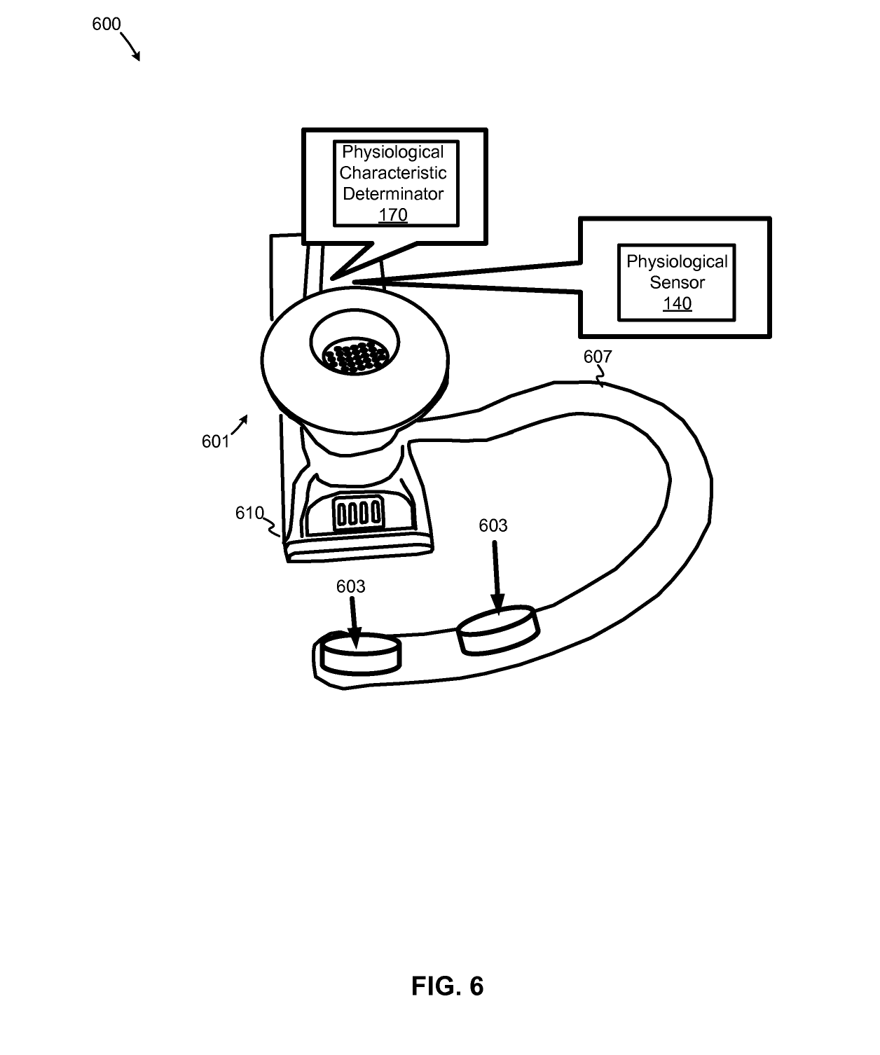

FIG. 6 depicts another example of an ear-related device/implement configured to provide for sensor terminals to sense physiological characteristics, according to some embodiments. Diagram 600 depicts ear-related device/implement 610 including an earbud 601 that can include one or more sensor terminals 603 disposed on or at a portion of an ear loop 607. In some examples, sensor terminals 603 are conductive and are configured to apply and/or receive a bioimpedance signal. Such a signal can be received by ear-related device/implement 610 which includes at least physiological sensor 140. In some examples, sensor terminal 603 can be a piezoelectric transducer or related structures. Thus, physiological sensor 140 can be a bioimpedance sensor or an acoustic sensor, such as a piezoelectric sensor. In some examples of physiological characteristic determinator 170 can be disposed in ear-related device/implement 610, but can also be disposed in any other device, in communication with ear-related device/implement 610, such as a mobile device or phone (not shown). In some examples, the portion of ear loop 607 is inserted behind an ear, whereby one or more sensor terminal 603s can be positioned adjacent to or in contact with tissue behind the ear. The loop portion 607 provides, at least in one example, a force via ear loop 607 to apply sensor terminals 603 to tissue.

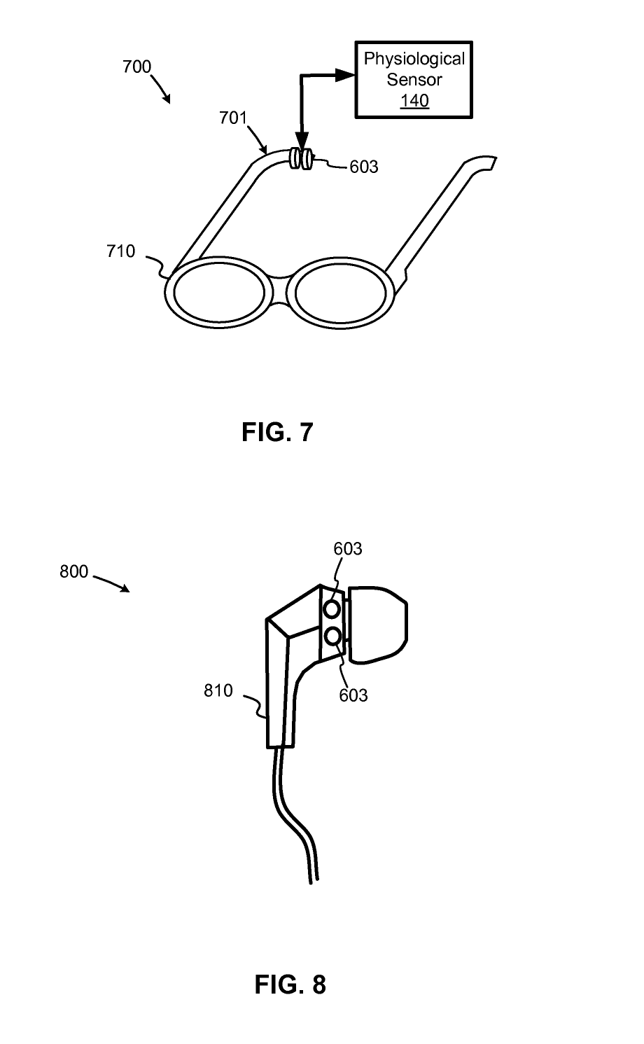

FIG. 7 depicts another example of an ear-related device/implement configured to provide sensor terminals to sense physiological characteristics, according to some embodiments. Diagram 700 depicts ear-related device/implement 610 as an implement (e.g., eyewear) including sensor terminals 603 disposed on or adjacent a temple tip 701 of eyewear 710.

FIG. 8 depicts yet another example of an ear-related device/implement configured to provide sensor terminals to sense physiological characteristics, according some embodiments. Diagram 800 depicts an earbud 810 configured to be inserted into an ear canal for providing audio. Earbud 810 can include sensor terminal 603 that are configured to contact tissues of the ear, such as at the ear canal. Therefore, earbud 810 can be used for sensing physiological characteristics, according to various embodiments.

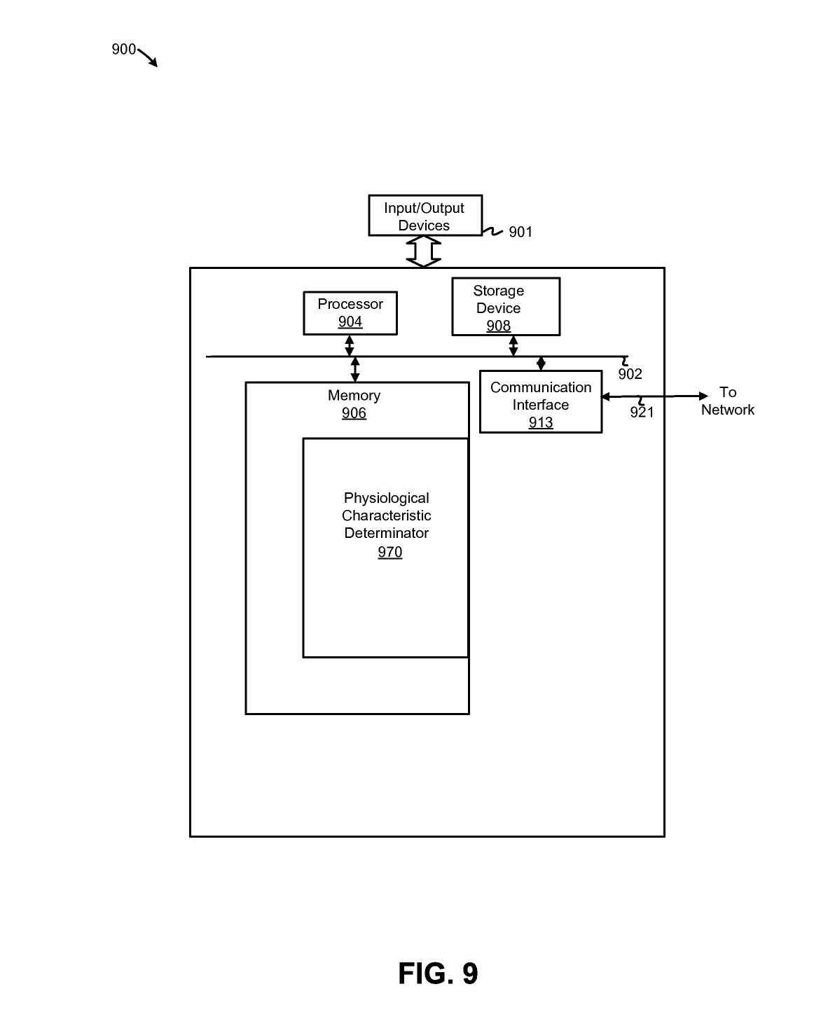

FIG. 9 illustrates an exemplary computing platform disposed in a configured to provide physiological characteristics in accordance with various embodiments. In some examples, computing platform 900 may be used to implement computer programs, applications, methods, processes, algorithms, or other software to perform the above-described techniques.

In some cases, computing platform can be disposed in an ear-related device/implement, a mobile computing device, or any other device.

Computing platform 900 includes a bus 902 or other communication mechanism for communicating information, which interconnects subsystems and devices, such as processor 904, system memory 906 (e.g., RAM, etc.), storage device 909 (e.g., ROM, etc.), a communication interface 913 (e.g., an Ethernet or wireless controller, a Bluetooth controller, etc.) to facilitate communications via a port on communication link 921 to communicate, for example, with a computing device, including mobile computing and/or communication devices with processors. Processor 904 can be implemented with one or more central processing units (“CPUs”), such as those manufactured by Intel® Corporation, or one or more virtual processors, as well as any combination of CPUs and virtual processors. Computing platform 900 exchanges data representing inputs and outputs via input-and-output devices 901, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 900 performs specific operations by processor 904 executing one or more sequences of one or more instructions stored in system memory 906, and computing platform 900 can be implemented in a client-server arrangement, peer-to-peer arrangement, or as any mobile computing device, including smart phones and the like. Such instructions or data may be read into system memory 906 from another computer readable medium, such as storage device 908. In some examples, hard-wired circuitry may be used in place of or in combination with software instructions for implementation. Instructions may be embedded in software or firmware. The term “computer readable medium” refers to any tangible medium that participates in providing instructions to processor 904 for execution. Such a medium may take many forms, including but not limited to, non-volatile media and volatile media. Non-volatile media includes, for example, optical or magnetic disks and the like. Volatile media includes dynamic memory, such as system memory 906.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 902 for transmitting a computer data signal.

In some examples, execution of the sequences of instructions may be performed by computing platform 900. According to some examples, computing platform 900 can be coupled by communication link 921 (e.g., a wired network, such as LAN, PSTN, or any wireless network) to any other processor to perform the sequence of instructions in coordination with (or asynchronous to) one another. Computing platform 900 may transmit and receive messages, data, and instructions, including program code (e.g., application code) through communication link 921 and communication interface 913. Received program code may be executed by processor 904 as it is received, and/or stored in memory 906 or other non-volatile storage for later execution.

In the example shown, system memory 906 can include various modules that include executable instructions to implement functionalities described herein. In the example shown, system memory 906 includes a physiological characteristic determinator 970, which can be configured to provide or consume outputs from one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

In some embodiments, a physiological sensor and/or physiological characteristic determinator can be in communication (e.g., wired or wirelessly) with a mobile device, such as a mobile phone or computing device, or can be disposed therein. In some cases, a mobile device, or any networked computing device (not shown) in communication with a physiological sensor and/or physiological characteristic determinator, can provide at least some of the structures and/or functions of any of the features described herein. As depicted in FIG. 1 and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in any of the figure can represent one or more algorithms. Or, at least one of the elements can represent a portion of logic including a portion of hardware configured to provide constituent structures and/or functionalities.

For example, a physiological sensor and/or physiological characteristic determinator, or any of their one or more components can be implemented in one or more computing devices (i.e., any mobile computing device, such as a wearable device, an audio device (such as headphones or a headset) or mobile phone, whether worn or carried) that include one or more processors configured to execute one or more algorithms in memory. Thus, at least some of the elements in FIG. 1 (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, a physiological sensor and/or physiological characteristic determinator, including one or more components, can be implemented in one or more computing devices that include one or more circuits. Thus, at least one of the elements in FIG. 1 (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method comprising:
receiving one or more signals from one or more sensor terminals, the one or more sensor terminals being disposed to contact portions of tissue at or in an ear of an organism, and ear-related device/implement being configured to apply one or more forces to the one or more sensor terminals to maintain contact with the portions of tissue;
sensing the one or more signals at the physiological sensor, the physiological sensor configured to sense a physiological characteristic of the organism; and
communicating data representing the one or more signals to a physiological characteristic determinator which is configured to determine a physiological characteristic of the organism.

2. The method of claim 1, wherein receiving the one or more signals from the one or more sensor terminals comprises:
receiving at least one bioimpedance signal.

3. The method of claim 2, wherein sensing the one or more signals at the physiological sensor comprises:
receiving the at least one bioimpedance signal at a bioimpedance sensor.

4. The method of claim 3, further comprising:
transmitting data representing the at least one bioimpedance signal via a wireless link to the physiological characteristic determinator.

5. The method of claim 4, further comprising:
generating a physiological characteristic signal that includes the data representing the physiological characteristic.

6. The method of claim 5, wherein generating the physiological characteristic signal comprises:
generating the physiological characteristic signal that includes the data representing one or more of a heart rate, a respiration rate, and a Mayer wave rate.

7. The method of claim 1, wherein receiving the one or more signals from the one or more sensor terminals comprises:
receiving at least one electric signal.

8. The method of claim 7, wherein sensing the one or more signals at the physiological sensor comprises:
receiving the at least one electric signal at a piezoelectric sensor.

9. The method of claim 8, further comprising:
generating a physiological characteristic signal that includes the data representing the physiological characteristic.

10. The method of claim 9, wherein generating the physiological characteristic signal comprises:
generating the physiological characteristic signal that includes the data representing one or more of a heart rate, a respiration rate, and a Mayer wave rate.

11. The method of claim 1, further comprising:
positioning at least one of the one or more sensor terminals adjacent to a region of tissue that includes a portion of a cymba conchae of the ear.

12. The method of claim 1, further comprising:
positioning at least one of the one or more sensor terminals adjacent to a region of tissue that includes a portion of a targus of the ear.

13. The method of claim 1, further comprising:
positioning at least one of the one or more sensor terminals at a region of tissue that includes a portion of a concha cavum of the ear.

14. The method of claim 1, further comprising:
positioning at least one of the one or more sensor terminals at a region of tissue that includes a portion of skin behind the ear.

15. A wearable device comprising:
one or more sensor terminals;
a physiological sensor coupled to the one or more sensor terminals, the physiological sensor configured to sense one or more signals originating at the one or more sensor terminals;
a radio frequency (“RF”) communications interface configured to communicate data representing the one or more signals; and
a processor configured to receive the one or more signals, and further configured to cause generation of data representing a physiological characteristic of the organism.

16. The wearable device of claim 15, further comprising:
an extension structure configured to position the one or more sensor terminals to contact portions of tissue at or in an ear of an organism, and is further configured to apply one or more forces to the one or more sensor terminals to maintain contact with the portions of tissue.

17. The wearable device of claim 15, wherein the physiological sensor comprises:
a bioimpedance sensor.

18. The wearable device of claim 15, wherein the physiological sensor comprises:
a piezoelectric sensor.

19. The wearable device of claim 15, wherein the physiological sensor comprises:
a skin surface microphone (“SSM”).

20. The wearable device of claim 15, wherein the wearable device constitutes a portion of one or more of a headset, eyewear, a mobile device and a wearable device.
